(19) United States
(12) Patent Application Publication
(10) Pub. No.: US 2024/0207479 A1
(43) Pub. Date: Jun. 27, 2024

(54) FRAGRANCE MACHINE

(71) Applicant: GUANGDONG YENIU TECHNOLOGY CO., LTD., DONGGUAN (CN)

(72) Inventor: JUN LONG, NANCHONG (CN)

(21) Appl. No.: 18/085,734

(22) Filed: Dec. 21, 2022

(51) Int. Cl. A61L 9/14

(52) U.S. Cl. CPC A61L 9/14

(57) ABSTRACT

The present invention discloses a fragrance machine, including a housing and an atomizing assembly. A limiting pipe is fixed in the housing; the limiting pipe is communicated with atmosphere outside the housing; an atomizing pipe and a liquid storage bottle are inserted in the limiting pipe; an atomizing core is fixed on the atomizing pipe; through holes are formed on the atomizing pipe; the liquid storage bottle is fixed with the atomizing pipe; the atomizing core and the through holes are limited at an inner side of an opened end of the liquid storage bottle; the atomizing core is provided with a liquid spray hole and an air spray hole which are arranged perpendicularly; the liquid spray hole is communicated with the interior of the liquid storage bottle; an air pump is communicated with the air spray hole; and a power supply is electrically connected with the air pump.

### TECHNICAL FIELD

The present invention relates to the field of atomization devices, and more particularly to a fragrance machine.

### BACKGROUND

There are three conventional modes of liquid atomization. One mode is a heating mode, i.e., the liquid is heated and then evaporated. Another mode is a mode of ultrasonic atomization, i.e., the liquid is atomized ultrasonically. The last mode is a mode of compression atomization, i.e., the compressed air flows through a small nozzle to form high-speed airflow and generate a negative pressure for driving the liquid or other fluids to be sprayed onto a barrier together, so that the liquid or other fluids splash around under high-speed impact, and the droplets become atomized particles to be sprayed out from an air outlet pipe.

One application field of liquid atomization is a fragrance machine. The existing fragrance machine mainly adopts the mode of ultrasonic atomization, which has low energy consumption, silence and good atomization effect. Due to different atomized liquids, the service life of ultrasonic atomizing piece is uncertain; and the atomizing piece needs to be wiped and cleaned regularly; otherwise the atomization effect will be affected. Some fragrance machines adopt the heating mode; this mode has good atomization effect, but high power consumption, so this mode is not suitable for portable fragrance machines. Some fragrance machines adopt the mode of compression atomization in the prior art; this mode has low energy consumption, good atomization effect and simple maintenance; however, the atomization effect is directly affected by the design of an atomization structure; and if the design of the atomization structure is unreasonable, the atomization performance cannot be exerted.

In addition, the fragrance liquid in the fragrance machine needs to be supplemented regularly, so it is required that a liquid storage bottle is easy to be disassembled and assembled while ensuring attractive appearance of the fragrance machine.

Therefore, how to provide a fragrance machine capable of solving the above problems is an urgent problem for those skilled in the art.

### SUMMARY

In view of this, the present invention provides a fragrance machine.

To achieve the above purpose, the following technical solution is adopted in the present invention:

A fragrance machine includes:
a housing, wherein a limiting pipe penetrating a lower end of the housing is fixed in the housing; a pipe length direction of the limiting pipe is the same as a length direction of the housing; a mounting hole is formed at an upper end of the housing; the limiting pipe is corresponding to and communicated with the mounting hole; an inner pipe wall of the limiting pipe is provided with a limiting groove arranged along the pipe length direction of the limiting pipe; and one end of the limiting groove penetrates through an upper end wall of the limiting pipe;
an atomizing assembly, wherein the atomizing assembly includes an atomizing pipe, a liquid storage bottle, limiting strips, an atomizing core, an air guide pipe, an air pump, a power supply, an elastic plug and an insertion pipe; one end of the atomizing pipe is closed; and the atomizing pipe and the liquid storage bottle are detachably inserted in the limiting pipe from top to bottom sequentially. The limiting strips are fixed on an outer pipe wall of the atomizing pipe; and the limiting strips can be embedded in the limiting groove. An opened end of the atomizing pipe is arranged close to the mounting hole; a closed end of the atomizing pipe is fixed with the atomizing core; the closed end of the atomizing pipe is provided with through holes; the opened end of the liquid storage bottle is detachably fixed with the closed end of the atomizing pipe; and the atomizing core and the through holes are all limited at an inner side of the opened end of the liquid storage bottle. The atomizing core is provided with a liquid spray hole and an air spray hole; a liquid inlet end of the liquid spray hole is communicated with the interior of the liquid storage bottle; a hole center line of the liquid spray hole is perpendicular and coplanar with that of the air spray hole; and a spray direction of the liquid spray hole points to the hole center line of the air spray hole. A pipe wall of the atomizing pipe is perforated and inserted with an elastic plug; a connecting hole is formed in the elastic plug; an air inlet channel is integrally formed in the atomizing pipe; and two ends of the air inlet channel are respectively communicated with the air spray hole and the connecting hole. The insertion pipe is fixed on the outer pipe wall of the limiting pipe; the insertion pipe penetrates through the pipe wall of the limiting pipe; the air guide pipe is respectively communicated with the insertion pipe and an air outlet end of the air pump; the elastic plug can be tightly abutted against the inner wall of the limiting pipe; and the connecting hole can be corresponding to and communicated with the insertion pipe. Both the air pump and the power supply are mounted in the housing; and the power supply is electrically connected with the air pump.

According to the above technical solution, compared with the prior art, the present invention discloses a fragrance machine; the atomizing pipe and the liquid storage bottle in the present invention are detachably connected with each other, and are detachably inserted in the limiting pipe, so that the liquid storage bottle is easy to be disassembled and assembled, and an operator can conveniently supplement liquid to the fragrance machine. The design of the elastic plug and the insertion pipe ensures that relatively high connection airtightness is still kept between the air pump and the air spray hole after the liquid storage bottle and the atomizing pipe are frequently disassembled and assembled. Through the arrangement of the limiting groove and the limiting strips, the connecting hole can be correctly aligned and communicated with the insertion pipe every time the liquid storage bottle is disassembled and assembled. The structural design of the air nozzle and the liquid nozzle ensures that the fragrance machine has a good atomization effect.

Preferably, a blind mounting hole is formed at the closed end of the atomizing pipe; the blind mounting hole is arranged adjacent to the through holes; and the air inlet channel is communicated with the blind mounting hole. The atomizing core includes a connecting pipe, an air nozzle, a mounting block, a liquid inlet pipe and a hose. The connecting pipe is embedded into the blind mounting hole; the air nozzle is tightly embedded into the connecting pipe; the air nozzle is provided with an air spray hole; and the hole center line of the air spray hole coincides with a pipe center line of the atomizing pipe. The mounting block is fixed at one end of the connecting pipe away from the blind mounting hole; and one side of the mounting block coincides with the hole center line of the air spray hole and is provided with a liquid spray hole. The liquid inlet pipe is integrally formed on the mounting block; and the liquid inlet pipe is limited in the liquid storage bottle and communicated with the liquid spray hole. One end of the hose is sleeved on the liquid inlet pipe; and the other end of the hose is abutted against a bottle bottom wall of the liquid storage bottle. The arrangement ensures that the fragrance machine has a good atomization effect.

Preferably, a side of the mounting block, which coincides with the hole center line of the air spray hole, is provided with a splayed open slot. A small opening end of the open slot points to the liquid inlet pipe; a diversion block is integrally formed in the center of a slot bottom wall of the open slot; and the liquid spray hole is formed in the diversion block. The above design makes the spray direction of the air spray hole more concentrated, thereby further improving the atomization effect of the fragrance machine.

Preferably, the atomizing assembly further includes a limiting piece, a pressing plate and an air inlet pipe. The limiting piece is a box body with a hollow interior and two opened ends; a length direction of the limiting piece is perpendicular to that of the limiting pipe; one end of the limiting piece is fixed with the outer side wall of the limiting pipe; the other end of the limiting piece is fixed with the pressing plate; the air guide pipe is limited in the limiting piece; one end of the air guide pipe away from the insertion pipe is closed; and the closed end of the air guide pipe is tightly abutted against the pressing plate. A long side wall of the limiting piece is provided with a gap; an air inlet pipe communicated with the interior of the air guide pipe is integrally formed on the outer side wall of the air guide pipe; and the air inlet pipe is fixed and communicated with the air outlet end of the air pump after passing through the gap. The arrangement ensures that the air guide pipe can be reliably connected with the insertion pipe to prevent air leakage.

Preferably, the atomizing assembly further includes a switch, a clamping piece and a charging interface; the switch is mounted at the upper end of the housing and electrically connected between the air pump and the power supply in series. The clamping piece is fixed on the outer side wall of the limiting pipe; the power supply is clamped on the clamping piece; the side wall of the housing is perforated and provided with a charging interface; and the charging interface is electrically connected with the power supply. The air pump can be controlled by the switch; the power supply can be reliably fixed with the limiting pipe through the clamping piece; and the power supply is a rechargeable battery, so that the fragrance machine is portable.

Preferably, the atomizing assembly further includes a first cover plate, a mist outlet pipe and a second cover plate. The opened end of the atomizing pipe is blocked by the first cover plate; the first cover plate is perforated in the center and vertically inserted with the mist outlet pipe; the first cover plate and the second cover plate are coaxially arranged; the first cover plate is limited in a region limited by an outer contour of the second cover plate; the second cover plate is tightly embedded in the mounting hole; and the second cover plate is perforated in the center and sleeved with the mist outlet pipe. The second cover plate not only can block the mounting hole, but also can ensure that the atomized air can be discharged from the mist outlet pipe.

### BRIEF DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely embodiments of the present invention, and for those ordinary skilled in the art, other drawings can also be obtained according to the drawings without contributing creative labor.

FIG. 1 is an overall axonometric view of a fragrance machine;

FIG. 2 is a partial axonometric view 1 of a fragrance machine;

FIG. 3 is a partial axonometric view 2 of a fragrance machine;

FIG. 4 is a partial axonometric view 3 of a fragrance machine;

FIG. 5 is a partial axonometric view 4 of a fragrance machine;

FIG. 6 is a partial axonometric view 5 of a fragrance machine;

FIG. 7 is a partial axonometric view 6 of a fragrance machine;

FIG. 8 is a partial axonometric view 7 of a fragrance machine;

FIG. 9 is a partial axonometric view 8 of a fragrance machine; and

FIG. 10 is an axonometric view of an elastic plug in a fragrance machine.

### IN THE FIGURES

1 outer cylinder; 2 upper cover; 3 lower cover; 4 limiting pipe; 40 limiting groove; 5 atomizing pipe; 50 through hole; 51 air inlet channel; 52 blind mounting hole; 6 liquid storage bottle; 7 limiting strip; 8 connecting pipe; 9 air nozzle; 90 air spray hole; 10 mounting block; 100 liquid spray hole; 101 open slot; 102 diversion block; 11 liquid inlet pipe; 12 hose; 13 air guide pipe; 14 air pump; 15 power supply; 16 limiting piece; 17 pressing plate; 18 air inlet pipe; 19 control circuit board; 20 mist volume switching and adjusting key; 21 timing key; 22 timing indicator light; 23 mist output volume indicator light; 24 clamping piece; 25 charging interface; 26 first cover plate; 27 mist outlet pipe; 28 second cover plate; 29 elastic plug; 290 connecting hole; 30 insertion pipe; and 31 light guide piece.

### DETAILED DESCRIPTION OF THE EMBODIMENTS

The technical solutions in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

The present invention discloses a fragrance machine. An atomizing pipe 5 and a liquid storage bottle 6 in the present invention are detachably connected with each other, and are detachably inserted in a limiting pipe 4, so that the liquid storage bottle 6 is easy to be disassembled and assembled while the overall appearance and structural compactness of the fragrance machine is ensured, and an operator can conveniently supplement liquid to the fragrance machine. The design of an elastic plug 29 and an insertion pipe 30 ensures that relatively high connection airtightness is still kept between the air pump 14 and the air spray hole 90 after the liquid storage bottle 6 and the atomizing pipe 5 are frequently disassembled and assembled. Through the arrangement of a limiting groove 40 and limiting strips 7, the connecting hole 290 can be correctly aligned and communicated with the insertion pipe 30 every time the liquid storage bottle 6 is disassembled and assembled. The structural design of an air nozzle 9, a liquid nozzle, an open slot 101, a diversion block 102 and the like ensures that the fragrance machine has a good atomization effect.

### EMBODIMENT

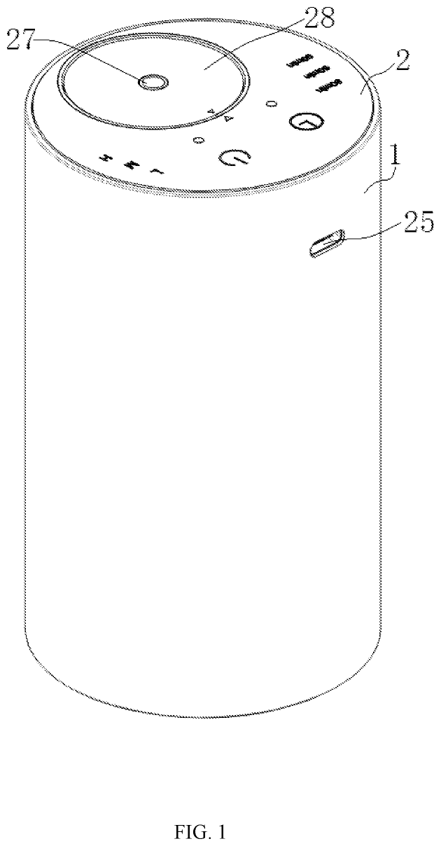

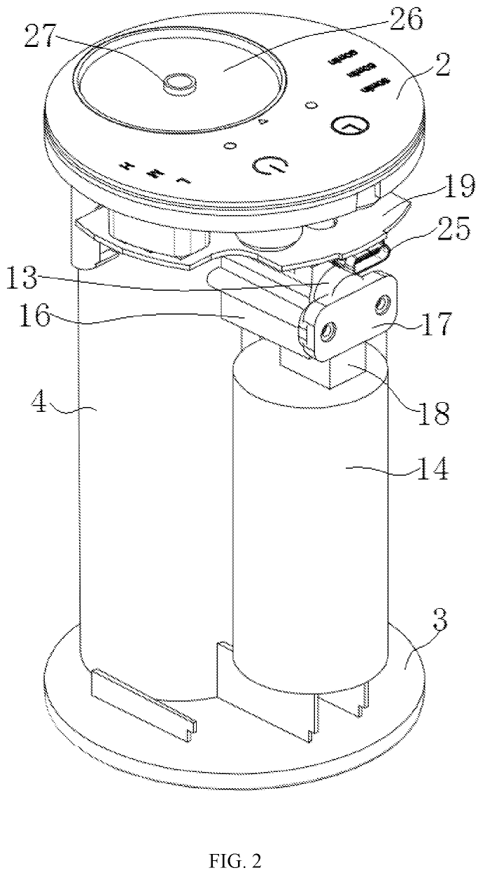

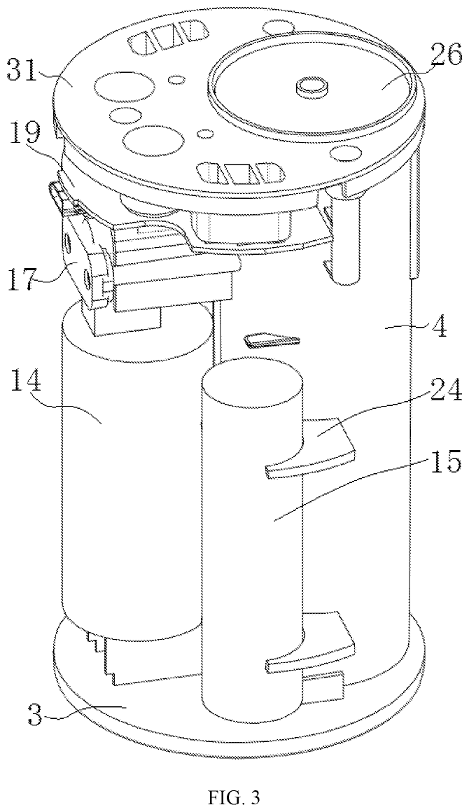

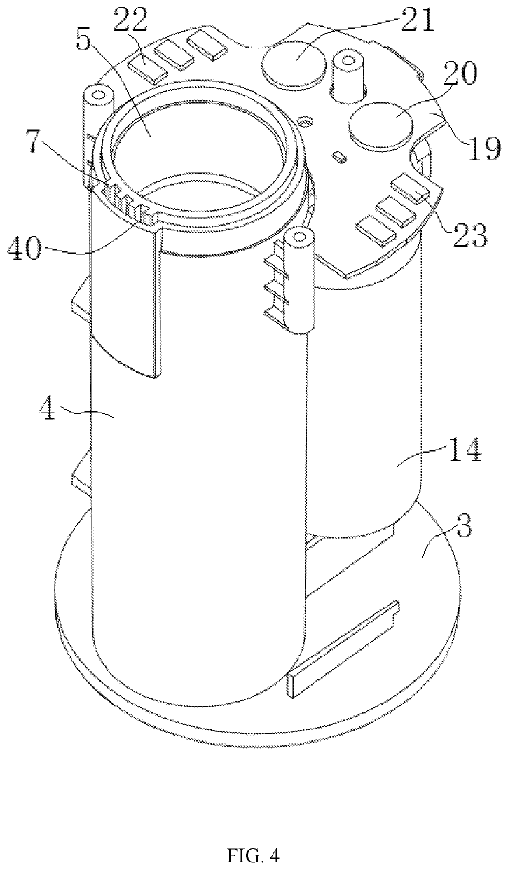

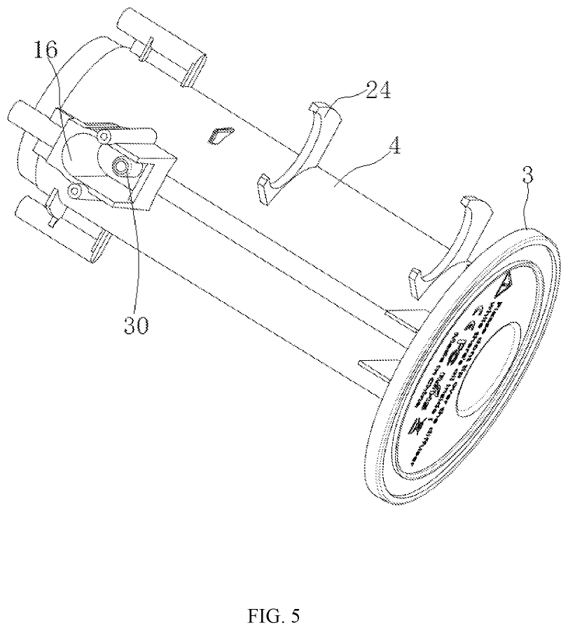

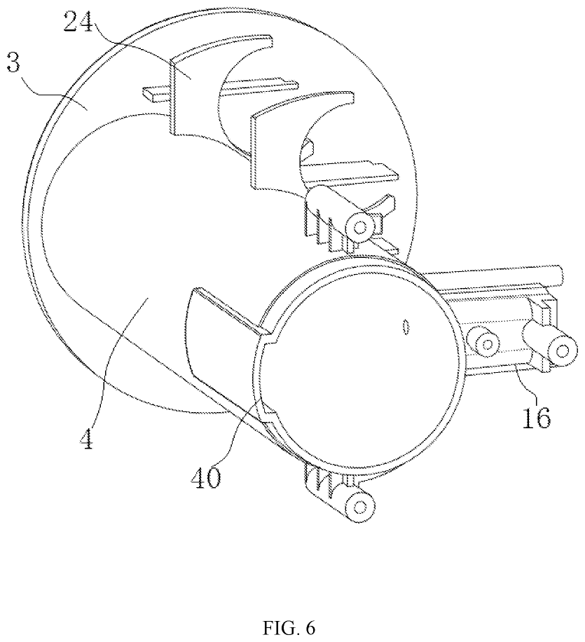

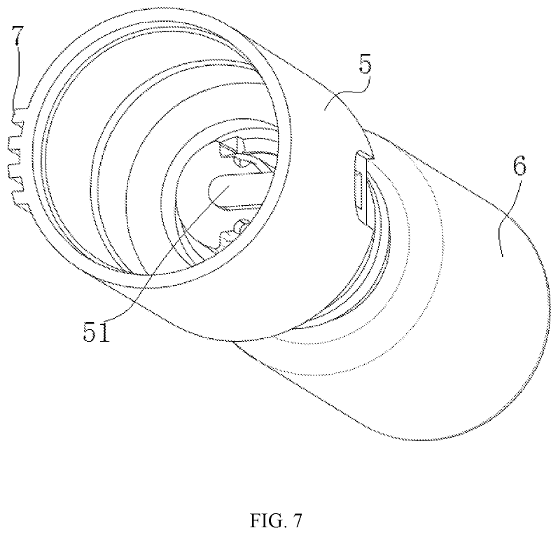

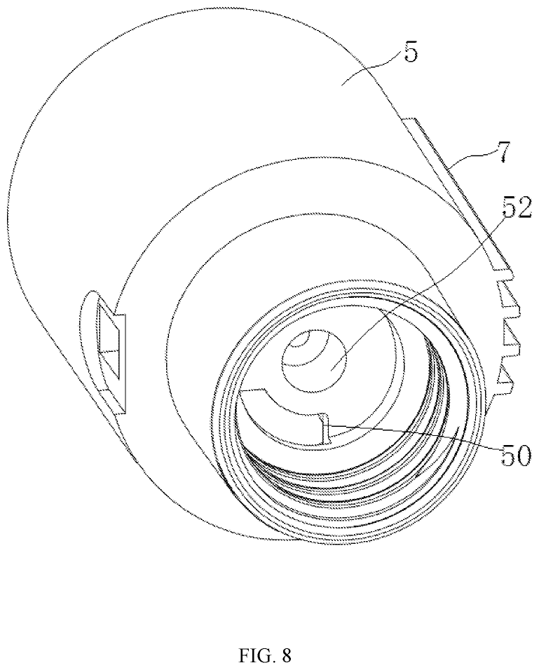

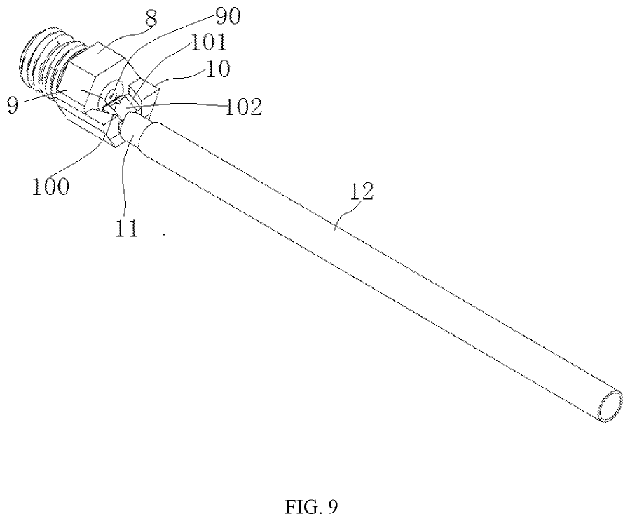

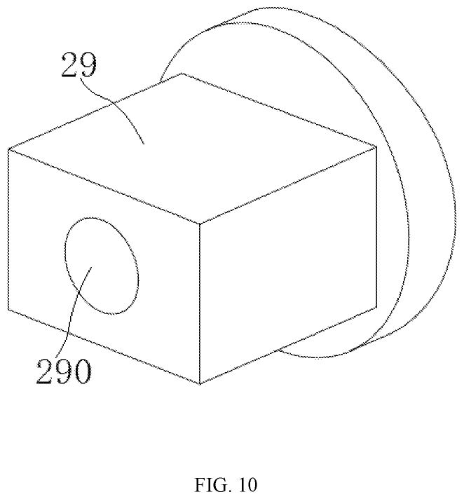

Referring to FIGS. 1-10 for the overall and partial structural schematic diagrams of an embodiment of the present invention, the present invention specifically discloses a fragrance machine, including:

a housing and an atomizing assembly, wherein the housing includes an outer cylinder 1, an upper cover 2 and a lower cover 3. An upper end and a lower end of the outer cylinder 1 are respectively blocked with a circular upper cover 2 and a circular lower cover 3; the limiting pipe 4 is arranged in the outer cylinder 1; a pipe length direction of the limiting pipe 4 is the same as a cylinder length direction of the outer cylinder 1; a lower end of the limiting pipe 4 is fixed with the lower cover 3; and a circular hole communicated with the limiting pipe 4 is formed in the lower cover 3. The circular hole is limited to an inner side of the limiting pipe 4; a circular mounting hole is formed in the upper cover 2; the limiting pipe 4 is corresponding to and communicated with the mounting hole; and a hole diameter of the mounting hole is greater than a pipe inner diameter of the limiting pipe 4. An inner pipe wall of the limiting pipe 4 is provided with a limiting groove 40 arranged along the pipe length direction of the limiting pipe 4; and one end of the limiting groove 40 penetrates through an upper end wall of the limiting pipe 4.

The atomizing assembly includes an atomizing pipe 5, a liquid storage bottle 6, limiting strips 7, an atomizing core, an air guide pipe 13, an air pump 14 and a power supply 15.

One end of the atomizing pipe 5 is opened; and the other end is closed. The atomizing pipe 5 and the liquid storage bottle 6 are detachably inserted in the limiting pipe 4 from top to bottom sequentially; and the liquid storage bottle 6 is filled with liquid.

A plurality of limiting strips 7 are fixed on an outer pipe wall of the atomizing pipe 5; and the plurality of limiting strips 7 can be simultaneously embedded in the limiting groove 40. A strip length direction of the limiting strips 7 is the same as a pipe length direction of the atomizing pipe 5; and one end of the limiting strips 7 can be abutted against one end wall of the limiting groove 40.

An opened end of the atomizing pipe 5 is arranged close to the mounting hole; a closed end of the atomizing pipe 5 is fixed with the atomizing core; the closed end of the atomizing pipe 5 is provided with a plurality of through holes 50; the opened end of the liquid storage bottle 6 is detachably fixed with the closed end of the atomizing pipe 5; and the atomizing core and the plurality of through holes 50 are all limited at an inner side of the opened end of the liquid storage bottle 6.

The atomizing core is provided with a liquid spray hole 100 and an air spray hole 90; a liquid inlet end of the liquid spray hole 100 is communicated with the interior of the liquid storage bottle 6; a hole center line of the liquid spray hole 100 is perpendicular and coplanar with that of the air spray hole 90; and a spray direction of the liquid spray hole 100 points to the hole center line of the air spray hole 90.

A pipe wall of the atomizing pipe 5 is perforated and inserted with an elastic plug 29; a connecting hole 290 is formed in the elastic plug 29; an air inlet channel 51 is integrally formed in the atomizing pipe 5; and two ends of the air inlet channel 51 are respectively communicated with the air spray hole 90 and the connecting hole 290. The insertion pipe 30 is fixed on the outer pipe wall of the limiting pipe 4; the insertion pipe 30 penetrates through the pipe wall of the limiting pipe 4; the air guide pipe 13 is respectively communicated with the insertion pipe 30 and an air outlet end of the air pump 14; the elastic plug 29 can be tightly abutted against the inner wall of the limiting pipe 4; and the connecting hole 290 can be corresponding to and communicated with the insertion pipe 30.

Both the air pump 14 and the power supply 15 are mounted in the housing; the power supply 15 is electrically connected with the air pump 14; and the air pump 14 is fixed with the outer wall of the limiting pipe 4.

A blind mounting hole 52 is formed at the closed end of the atomizing pipe 5; the blind mounting hole 52 is arranged adjacent to the through holes 50; and the air inlet channel 51 is communicated with the blind mounting hole 52.

The atomizing core includes a connecting pipe 8, an air nozzle 9, a mounting block 10, a liquid inlet pipe 11 and a hose 12. The connecting pipe 8 is embedded into the blind mounting hole 52; the air nozzle 9 is tightly embedded into the connecting pipe 8; the air nozzle 9 is provided with the air spray hole 90; and the hole center line of the air spray hole 90 coincides with a pipe center line of the atomizing pipe 5. The mounting block 10 is fixed at one end of the connecting pipe 8 away from the blind mounting hole 52; and one side of the mounting block 10 coincides with the hole center line of the air spray hole 90 and is provided with a liquid spray hole 100. The liquid inlet pipe 11 is integrally formed on the mounting block 10; and the liquid inlet pipe 11 is limited in the liquid storage bottle 6 and communicated with the liquid spray hole 100. One end of the hose 12 is sleeved on the liquid inlet pipe 11; and the other end of the hose 12 is abutted against a bottle bottom wall of the liquid storage bottle 6.

A side of the mounting block 10, which coincides with the hole center line of the air spray hole 90, is provided with a splayed open slot 101. A small opening end of the open slot 101 points to the liquid inlet pipe 11; a diversion block 102 is integrally formed in the center of a slot bottom wall of the open slot 101; and the liquid spray hole 100 is formed in the diversion block 102.

Further specifically, the atomizing assembly further includes a limiting piece 16, a pressing plate 17 and an air inlet pipe 18. The limiting piece 16 is a box body with a hollow interior and two opened ends; and a length direction of the limiting piece 16 is perpendicular to that of the limiting pipe 4. One end of the limiting piece 16 is fixed with the outer side wall of the limiting pipe 4; the other end of the limiting piece 16 is fixed with the pressing plate 17 in a screw connection manner; and the air guide pipe 13 is limited in the limiting piece 16. One end of the air guide pipe 13 away from the insertion pipe 30 is closed; and the closed end of the air guide pipe 13 is tightly abutted against the pressing plate 17. The pressing plate 17 can be abutted against the air guide pipe 13 to ensure that the air guide pipe 13 can be reliably connected with the insertion pipe 30.

A long side wall of the limiting piece 16 is provided with a gap; an air inlet pipe 18 communicated with the interior of the air guide pipe 13 is integrally formed on the outer side wall of the air guide pipe 13; and the air inlet pipe 18 is fixed and communicated with the air outlet end of the air pump 14 after passing through the gap.

Further specifically, the atomizing assembly further includes a switch, a clamping piece 24 and a charging interface 25. The switch includes a control circuit board 19, a mist volume switching and adjusting key 20, a timing key 21, a timing indicator light 22 and a mist output volume indicator light 23. The control circuit board 19 is arranged below the upper cover 2 and fixed with the limiting pipe 4; the upper cover 2 is made of light guide materials; and a light guide piece 31 is fixed between the control circuit board 19 and the upper cover 2. The light guide piece 31 can restrain light emitted by the timing indicator light 22 and the mist output volume indicator light 23, so that the light emitted by the timing indicator light 22 and the mist output volume indicator light 23 can be independently displayed on the upper cover 2 without interfering with each other. The control circuit board 19 is electrically connected with the air pump 14 and the power supply 15, respectively. The mist volume switching and adjusting key 20, the timing key 21, the timing indicator light 22 and the mist output volume indicator light 23 are electrically integrated on the control circuit board 19; and both the mist volume switching and adjusting key 20 and the timing key 21 are touch keys.

The clamping piece 24 is fixed on the outer side wall of the limiting pipe 4; the power supply 15 is clamped on the clamping piece 24; and the power supply 15 in the present embodiment is a rechargeable battery. The side wall of the housing is perforated and provided with a charging interface 25; and the charging interface 25 is electrically integrated on the control circuit board 19.

Further specifically, the atomizing assembly further includes a first cover plate 26, a mist outlet pipe 27 and a second cover plate 28. The opened end of the atomizing pipe 5 is blocked by a circular first cover plate 26; and the first cover plate 26 is perforated in the center and vertically inserted with the mist outlet pipe 27. The first cover plate 26 and a circular second cover plate 28 are coaxially arranged; the first cover plate 26 is limited in a region limited by an outer contour of the second cover plate 28, i.e., an outer diameter of the first cover plate 26 is smaller than that of the second cover plate 28; the second cover plate 28 is tightly embedded in the mounting hole; and the second cover plate 28 is perforated in the center and sleeved with the mist outlet pipe 27.

When the fragrance machine is in operation, the operator vertically arranges the outer cylinder 1 and then turns on the switch; and the air pump 14 starts pumping air into the liquid storage bottle 6. The air is sprayed out from the air spray hole 90 at high speed, to form a negative pressure near the air spray hole 90. The high-speed air sprayed out from the air spray hole 90 will be converged by the diversion block 102 and the open slot 101, so that a strong negative pressure will be generated near the liquid spray hole 100. The liquid spray hole 100 sucks the liquid in the liquid storage bottle 6 through the liquid inlet pipe 11 and the hose 12. While the air spray hole 90 continuously sprays air out, the liquid spray hole 100 will also spray liquid out due to the action of the negative pressure; and the liquid sprayed out from the liquid spray hole 100 will be rapidly atomized into tiny droplets and be scattered. Because the air spray hole 90 blows air into the liquid storage bottle 6, a positive pressure will be formed in the liquid storage bottle 6; and the air mixed with the atomized liquid will be discharged from the mist outlet pipe 17.

When the liquid storage bottle 6 needs to be filled with liquid, a user pushes out the liquid storage bottle 6 and the atomizing pipe 5 from the circular hole, then unscrews the liquid storage bottle 6 and the atomizing pipe 5, and supplements the liquid into the liquid storage bottle 6. After the liquid is supplemented, the liquid storage bottle 6 and the atomizing pipe 5 are screwed in; and then the liquid storage bottle 6 and the atomizing pipe 5 are sequentially inserted in the limiting pipe 4 from the mounting hole. Because the limiting groove 40 and the limiting strips 7 are provided, it is ensured that the elastic plug 29 is tightly abutted against the inner wall of the limiting pipe 4 after the liquid storage bottle 6 and the atomizing pipe 5 are inserted, and the connecting hole 290 can be corresponding to and communicated with the insertion pipe 30.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A fragrance machine, comprising:
a housing, wherein a limiting pipe (4) penetrating a lower end of the housing is fixed in the housing; a pipe length direction of the limiting pipe (4) is the same as a length direction of the housing; a mounting hole is formed at an upper end of the housing; the limiting pipe (4) is corresponding to and communicated with the mounting hole; an inner pipe wall of the limiting pipe (4) is provided with a limiting groove (40) arranged along the pipe length direction of the limiting pipe; and one end of the limiting groove (40) penetrates through an upper end wall of the limiting pipe (4);
an atomizing assembly, wherein the atomizing assembly comprises an atomizing pipe (5), a liquid storage bottle (6), limiting strips (7), an atomizing core, an air guide pipe (13), an air pump (14), a power supply (15), an elastic plug (29) and an insertion pipe (30); one end of the atomizing pipe (5) is closed, and the atomizing pipe and the liquid storage bottle (6) are detachably inserted in the limiting pipe (4) from top to bottom sequentially; the limiting strips (7) are fixed on an outer pipe wall of the atomizing pipe (5); the limiting strips (7) can be embedded in the limiting groove (40); an opened end of the atomizing pipe (5) is arranged close to the mounting hole; a closed end of the atomizing pipe (5) is fixed with the atomizing core; the closed end of the atomizing pipe (5) is provided with through holes (50); the opened end of the liquid storage bottle (6) is detachably fixed with the closed end of the atomizing pipe (5); the atomizing core and the through holes (50) are all limited at an inner side of the opened end of the liquid storage bottle (6); the atomizing core is provided with a liquid spray hole (100) and an air spray hole (90); a liquid inlet end of the liquid spray hole (100) is communicated with the interior of the liquid storage bottle (6); a hole center line of the liquid spray hole (100) is perpendicular and coplanar with a hole center line of the air spray hole (90); a spray direction of the liquid spray hole (100) points to the hole center line of the air spray hole (90); a pipe wall of the atomizing pipe (5) is perforated and inserted with an elastic plug (29); a connecting hole (290) is formed in the elastic plug (29); an air inlet channel (51) is integrally formed in the atomizing pipe (5); two ends of the air inlet channel (51) are respectively communicated with the air spray hole (90) and the connecting hole (290); the insertion pipe (30) is fixed on the outer pipe wall of the limiting pipe (4); the insertion pipe (30) penetrates through the pipe wall of the limiting pipe (4); the air guide pipe (13) is respectively communicated with the insertion pipe (30) and an air outlet end of the air pump (14); the elastic plug (29) can be tightly abutted against the inner wall of the limiting pipe (4); the connecting hole (290) can be corresponding to and communicated with the insertion pipe (30); both the air pump (14) and the power supply (15) are mounted in the housing; and the power supply (15) is electrically connected with the air pump (14).

2. The fragrance machine according to claim 1, wherein a blind mounting hole (52) is formed at the closed end of the atomizing pipe (5); the blind mounting hole (52) is arranged adjacent to the through holes (50); the air inlet channel (51) is communicated with the blind mounting hole (52); the atomizing core comprises a connecting pipe (8), an air nozzle (9), a mounting block (10), a liquid inlet pipe (11) and a hose (12); the connecting pipe (8) is embedded into the blind mounting hole (52); the air nozzle (9) is tightly embedded into the connecting pipe (8); the air nozzle (9) is provided with an air spray hole (90); the hole center line of the air spray hole (90) coincides with a pipe center line of the atomizing pipe (5); the mounting block (10) is fixed at one end of the connecting pipe (8) away from the blind mounting hole (52); one side of the mounting block (10) coincides with the hole center line of the air spray hole (90) and is provided with a liquid spray hole (100); the liquid inlet pipe (11) is integrally formed on the mounting block (10); the liquid inlet pipe (11) is limited in the liquid storage bottle (6) and communicated with the liquid spray hole (100); one end of the hose (12) is sleeved on the liquid inlet pipe (11); and the other end is abutted against a bottle bottom wall of the liquid storage bottle (6).

3. The fragrance machine according to claim 2, wherein a side of the mounting block (10), which coincides with the hole center line of the air spray hole (90), is provided with a splayed open slot (101); a small opening end of the open slot (101) points to the liquid inlet pipe (11); a diversion block (102) is integrally formed in the center of a slot bottom wall of the open slot (101); and the liquid spray hole (100) is formed in the diversion block (102).

4. The fragrance machine according to claim 1, wherein the atomizing assembly further comprises a limiting piece (16), a pressing plate (17) and an air inlet pipe (18); the limiting piece (16) is a box body with a hollow interior and two opened ends; a length direction of the limiting piece (16) is perpendicular to a length direction of the limiting pipe (4); one end of the limiting piece (16) is fixed with the outer side wall of the limiting pipe (4); the other end of the limiting piece (16) is fixed with the pressing plate (17); the air guide pipe (13) is limited in the limiting piece (16); one end of the air guide pipe (13) away from the insertion pipe (30) is closed; the closed end of the air guide pipe (13) is tightly abutted against the pressing plate (17); a long side wall of the limiting piece (16) is provided with a gap; an air inlet pipe (18) communicated with the interior of the air guide pipe is integrally formed on the outer side wall of the air guide pipe (13); and the air inlet pipe (18) is fixed and communicated with the air outlet end of the air pump (14) after passing through the gap.

5. The fragrance machine according to claim 1, wherein the atomizing assembly further comprises a switch, a clamping piece (24) and a charging interface (25); the switch is mounted at the upper end of the housing and electrically connected between the air pump (14) and the power supply (15) in series; the clamping piece (24) is fixed on the outer side wall of the limiting pipe (4); the power supply (15) is clamped on the clamping piece (24); the side wall of the housing is perforated and provided with a charging interface (25); and the charging interface (25) is electrically connected with the power supply (15).

6. The fragrance machine according to claim 1, wherein the atomizing assembly further comprises a first cover plate (26), a mist outlet pipe (27) and a second cover plate (28); the opened end of the atomizing pipe (5) is blocked by the first cover plate (26); the first cover plate (26) is perforated in the center and vertically inserted with the mist outlet pipe (27); the first cover plate (26) and the second cover plate (28) are coaxially arranged; the first cover plate (26) is limited in a region limited by an outer contour of the second cover plate (28); the second cover plate (28) is tightly embedded in the mounting hole; and the second cover plate (28) is perforated in the center and sleeved with the mist outlet pipe (27).
